(19) United States
(12) Patent Application Publication
(10) Pub. No.: US 2004/0193022 A1
(43) Pub. Date: Sep. 30, 2004

(54) Diagnostic support system and diagnostic support program

(75) Inventors: Masanobu Torii, Kawasaki (JP); Shouta Watanabe, Kawasaki (JP); Tatsuya Suwa, Kawasaki (JP); Hiromi Kato, Kawasaki (JP)

Correspondence Address: STAAS & HALSEY LLP, SUITE 700, 1201 NEW YORK AVENUE, N.W., WASHINGTON, DC, 20005, US

(73) Assignee: Fujitsu Limited, Kawasaki (JP)

(21) Appl. No.: 10/784,931

(22) Filed: Feb. 25, 2004

(30) Foreign Application Priority Data
Mar. 24, 2003 (JP) 2003-081433

(51) Int. Cl. A61B 5/00

(52) U.S. Cl. 600/300; 128/920

(57) ABSTRACT

A case database is stored with values in a plurality of fields with respect to symptoms of past patients. A CPU, when new patient data are inputted, calculates a degree of similarity of each piece of case data to the new patient data as a total sum of a values obtained by weighting a difference between each value in the case data and its corresponding value in the new patient data in accordance with an influence degree of the value in the patient data, obtains a degree of similarity of each disease name as a total sum of degrees of similarity in the all pieces of case data having this disease name, and displays the disease name exhibiting the maximum degree of similarity together with the value in the new patient data that exhibits the maximum influence degree used for calculating the degree of similarity.

### BACKGROUND OF THE INVENTION

1. Field of the Invention

The present invention relates to a diagnostic support system for analogizing a name of a-disease on the basis of a symptom of a patient and a result of examination and displaying the disease name on such an occasion that a doctor attends a patient, and to a diagnostic support program for making a computer function as this type of diagnostic support system.

2. Description of the Prior Art

A doctor judges a name of a disease from which the patient suffers by collating statistic and dynamic information about the patient such as a disease history, a subjective symptom, results of measurements of a body temperature, a blood pressure and brain waves, results of pathological examinations for a subject and images by x-ray photograph, etc. with a variety of patient medical records accumulated in the past and with literatures such as medical books, medical thesis and so on; and determines a therapeutic method suited to this wound or disease. Accordingly, a validity of the diagnosis depends on a capacity of searching for a large quantity of literatures in addition to an experience, an amount of knowledge and skill of the individual doctor. Hence, there have hitherto been proposed a variety of systems utilized for collating the aforementioned statistic and dynamic information about the patient with the literatures. For example, a computerized medical record system is that the statistic information (a profile of the patient, examination values) recorded on a conventional paper medical sheet and the dynamic information (a complaint of the patient, an observation of the examination, a judgement by the doctor, a treatment plan) are computerized in a electronic data format and stored on a database. Besides, Japanese Patent Application Laid-open Publication No.8-26568 discloses a system for presuming a name of a disease on the basis of a symptom of the patient and examination results.

The computerizes medical record system described above is not, however, superior to a function of a normal database system, and hence a doctor himself or herself must set retrieval conditions such as a keyword, a range of each of the examination values, etc. when executing retrieval. Besides, even when past medical records coincident with the retrieval conditions could be extracted, if the extracted records were just for one case, or if none of disease names therein were coincident with one another in spite of a plurality of cases being extracted, this implies that the name of the disease of the patient was neither specified nor presumed. According to the system disclosed in Japanese Patent Application Laid-open Publication given above, the disease name is presumed, however, pieces of information presented to the doctor were just the disease name and a degree of credibility. Therefore, the doctor does not recognize what reason the disease name was presumed for, and must therefore verify this disease name by collating pieces of information that serve as a basis for the presumption with one another. Especially in a case where a plurality of disease names are presumed to almost the same degree of credibility, the doctor must check each of the grounds on which the respective disease names were presumed and must verify which ground the validity was obtained from. Thus, none of the systems that have been proposed so far were capable of meeting requirements of EBM (Evidence-Based Medicine: which means an effective and high-grade medical practice based on an optimal ground just when diagnosing an individual patient) in recent years.

### SUMMARY OF THE INVENTION

It is an object of the present invention to provide a diagnostic support system for presuming a name of a disease from which a relevant patient suffers in a way that collates with case data accumulated in the past with respect to symptoms of patients and displaying the presumed disease name together with a ground for the presumption, and a diagnostic support program making a computer function as this type of medical decision support system.

A processing device of the computer configuring the diagnostic support system of the present invention devised for solving the above problems and the computer operating based on the diagnostic support program of the present invention calculate, for every concrete values in respective fields in case data accumulated in a case database, an influence degree of the value contributing to determine a disease name; when. new patient data including value in some or all of the predetermined fields with respect to a symptom of a new patient are inputted via an input device, calculate a degree of similarity of each piece of case data to new patient data on the basis of values obtained for respective fields by weighting a difference between a value in each field of the case data and a value in its corresponding field of the new patient data with influence degree of that value in the new patient data; calculate, for every disease name, a degree of similarity of the disease name on the basis of degrees of similarity in all the case data having this disease name; and display on a display device a disease name exhibiting a maximum degree of similarity together with the value in the field in the new patient data of which influence degree is maximum among those used for calculating the degree of similarity.

Accordingly, when an operator (a doctor, etc.). inputs new patient data about the symptom of the new patient by use of the input device, the degree of similarity of each piece of case data to the new patient data is calculated taking into account the influence degree of every concrete value in each field of the new patient data, the degree of similarity of each disease name is calculated based on the calculated degree of similarity of each piece of case data, and the disease name exhibiting the maximum degree of similarity among those calculated is displayed as a presumed disease name on the display device. Together with this presumed disease name, the value exhibiting the maximum influence degree among the values in the respective fields of the new patient data which have been used for calculating the degrees of similarity, is displayed on the display device. Accordingly, the operator is able to know what ground the disease name has been presumed from.

Note that in addition to the value in the new patient data that exhibits the maximum influence degree, the values in the fields in a predetermined number of pieces of new patient data may be displayed in sequence from the value exhibiting the maximum influence degree together with the influence degree thereof on the display device according to the present invention. This scheme enables the operator to know in greater detail the ground which the disease name has been presumed from because of being able to know the values of which the influence degrees are the second or lower in rank.

Further, according to the present invention, if any piece of case data containing the disease name displayed on the display device contains the value substantially coincident with the value in the new patient data that is displayed on the display device, this field value may be displayed in a form corresponding to its fact. This scheme enables the operator to know an existence of a similar case having the value in the same field that has the presumed disease name and exhibits the large influence degree and to therefore have a confidence in the presumed disease name.

Moreover, according to the present invention, in a case where an instruction of displaying the similar case with respect to the disease name displayed on the display device is inputted through the input device, contents of a predetermined number of pieces of case data among those having that disease name may be displayed in sequence from the content exhibiting the maximum degree of similarity calculated. with this scheme adopted, the operator is able to know details of the similar cases in the past and is therefore able to easily verify a validity of the presumed disease name.

### BRIEF DESCRIPTION OF THE DRAWINGS

The invention will be described below in detail with reference to the accompanying drawings, in which:

FIG. 1 is a block diagram showing a hardware architecture of a computer network system by way of an embodiment of a diagnostic support system according to the present invention;

FIG. 2 is a table showing an outline of a data structure of a patient profile table constituting a computerized medical record database;

FIG. 3 is a table showing an outline of a data structure of an examination data table constituting the-computerized medical record database;

FIG. 4 is a table showing an outline of a data structure of an attending data table constituting the computerized medical record database;

FIG. 5 is a table showing an outline of a data structure of an analysis database;

FIG. 6 is a table showing an outline of a data structure of a case database;

FIG. 7 is a table showing an outline of a data structure of a medical treatment database;

FIG. 8 is a flowchart showing processes based on an analysis data creation program;

FIG. 9 is a flowchart showing processes based on a case data creation program;

FIG. 10 is a flowchart showing a cross-testing process subroutine executed in s13 in FIG. 9;

FIG. 11 is a flowchart showing the cross-testing process subroutine executed in s13 in FIG. 9;

FIG. 12 is a flowchart showing processes based on a medical treatment data creation program;

FIG. 13 is a flowchart showing processes based on a disease name prediction program;

FIG. 14 is a flowchart showing processes based on an information display program;

FIG. 15 is a diagram showing an input screen;

FIG. 16 is a diagram showing a disease confirmation screen;

FIG. 17 is a diagram showing a similar case screen;

FIG. 18 is a diagram showing a medical treatment process screen; and

FIG. 19 is a diagram showing a necessary expenditure screen.

### DESCRIPTION OF THE PREFERRED EMBODIMENT

An embodiment of a diagnostic support system according to the present invention will hereinafter be described with reference to the accompanying drawings.

The present embodiment is actualized, based on a conventional computerized medical record system, and by applying a MBR (Memory Based Reasoning) algorithm. This MBR is one of classification algorithms to the conventional system. The MBR is an algorithm of retrieving a plurality of cases similar to an unidentified thing as an object of classification from a great quantity of past data accumulated, and classifying the unknown case by a weighted majority decision for the plurality of the retrieved similar cases.

(Whole Architecture)

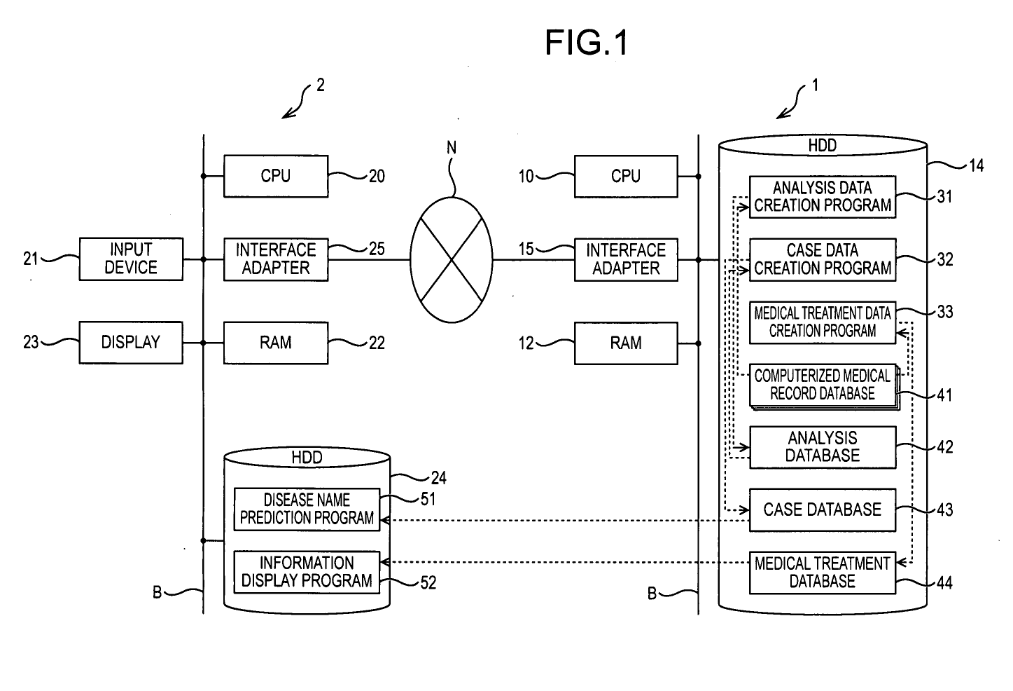

FIG. 1 is a block diagram showing an outline of an architecture of a computer network system by way of an embodiment of the diagnostic support system of the present invention. As shown in FIG. 1, this computer network system is configured of a single host computer 1 and a multiplicity of terminals 2 which are communicable with each other via a computer network (LAN: Local Area Network) N established within a hospital. This host computer 1 is a database server allocated in a library, etc. within the hospital, which is a developed version of a conventional host computer in the conventional computerized medical record system, to which a variety of programs 31 though 33 and various categories of databases 41 through 44 that will be described later on are added. Further, each of the terminals 2 is a computer allocated in a consulting room, a medical office and a doctor's office and is equivalent to the terminal configuring the conventional computerized medical record system additionally installed with a variety of programs 51, 52 that will hereinafter be explained.

(Host Computer)

The host computer 1 is constructed of a CPU (central Processing unit) 10 for controlling the whole device, and a RAM (Random Access memory) 12, a hard disk 14 and an interface adapter 15 which are connected via a bus B to this CPU 10. Among these components, the RAM 12 is a main memory device on which an operation area utilized by the CPU 10 is developed, and the interface adapter 15 is defined as a LAN card serving as an interface with the computer network N.

The hard disk 14 is a computer readable storage medium such as a hard disk stored with the variety of programs and the various categories of data. As explained above, this host computer 1 is the developed version of the host computer in the conventional computerized medical record system, and hence the hard disk 14 is preinstalled with a computerized medical record database 41 and an unillustrated computerized medical record data server program in addition to an unillustrated OS (Operating System) that supports a function for performing communications via the computer network N. The unillustrated computerized medical record data server program instructs the CPU 10 to format the data transmitted from the respective terminals 2 and to store the thus formatted data on the computerized medical record database 41, to retrieve the computerized medical record database 41 in response to a retrieval request that designates a retrieval condition sent from the terminal 2 and to respond a content of an extracted data record to the retrieval requester terminal 2.

The computerized medical record database 41 is a database for recording pieces of information which have hitherto been written on a paper medical sheet in a way that these pieces of information are divided into a plurality of tables linked to each other by using a patient ID as a key. The tables constituting this computerized medical record database 41 include a patient profile data table for recording individual information of each individual patient, an examination data table for recording examination data of each individual patient, an attending data table for recording a medical history of each individual patient, and so on.

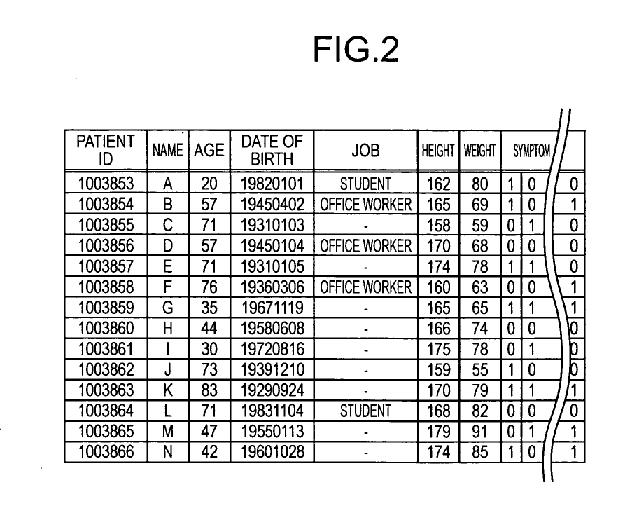

FIG. 2 is a table showing an example of a data structure of the patient profile data table. As shown in FIG. 2, this patient profile data table is structured of a plurality of records each created for every patient. Each record contains items (fields) such as a patient ID for uniquely identifying the patient, a name, an age, a date of patient's birth, a job, a height, a weight and a symptom of this patient. Note that numerical values into which, for example, a distinction whether there is a shivers or not, a distinction whether there is nausea or not, a distinction whether there is a stomachache or not, etc. are digitized, are recorded in the “symptom” field.

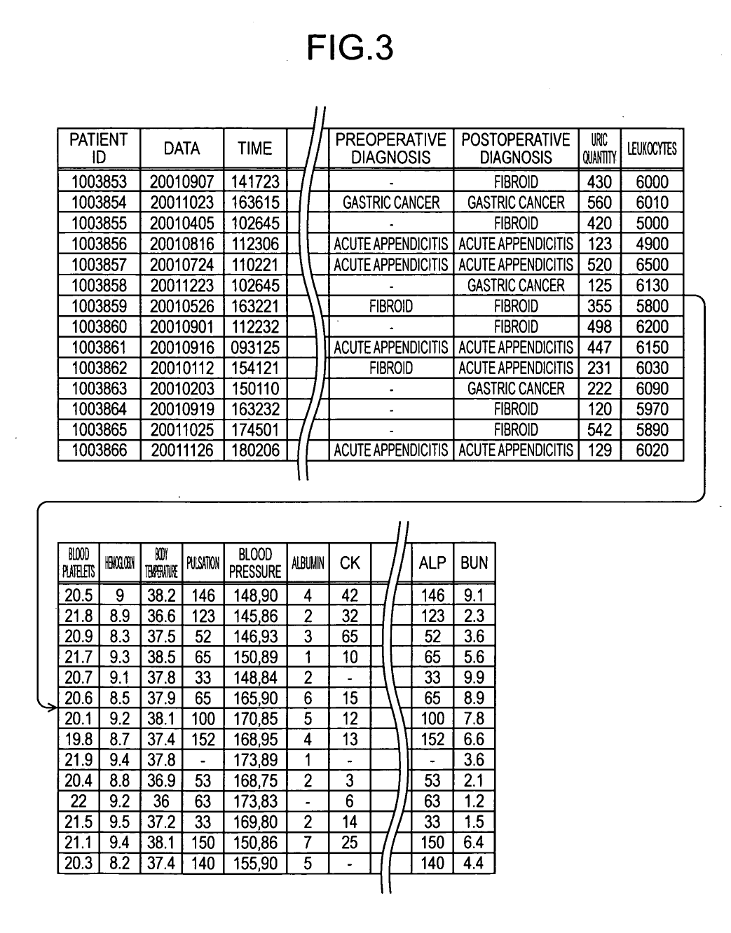

FIG. 3 is a table showing an example of a data structure of the examination data table (a preoperative examination data table) for recording a result of the examination before an operation among the various categories of data tables. As shown in FIG. 3, this examination data table is structured of a plurality of records each created for every patient. Each record contains items (fields) such as a patient ID of the patient, an examination executing date (DATA), an examination executing time (TIME), a disease name (a preoperative diagnosed disease name) diagnosed before an operation related to that examination, a disease name (a postoperative diagnosed disease name) established through the operation, and a variety of examination values (an uric quantity, leukocytes, blood platelets, hemoglobin, a body temperature, pulsation, a blood pressure, albumin, CK: creatine Kinase, ALP: Alkaline Phosphatase, BUN: Blood urea Nitrogen as a result of the examination.

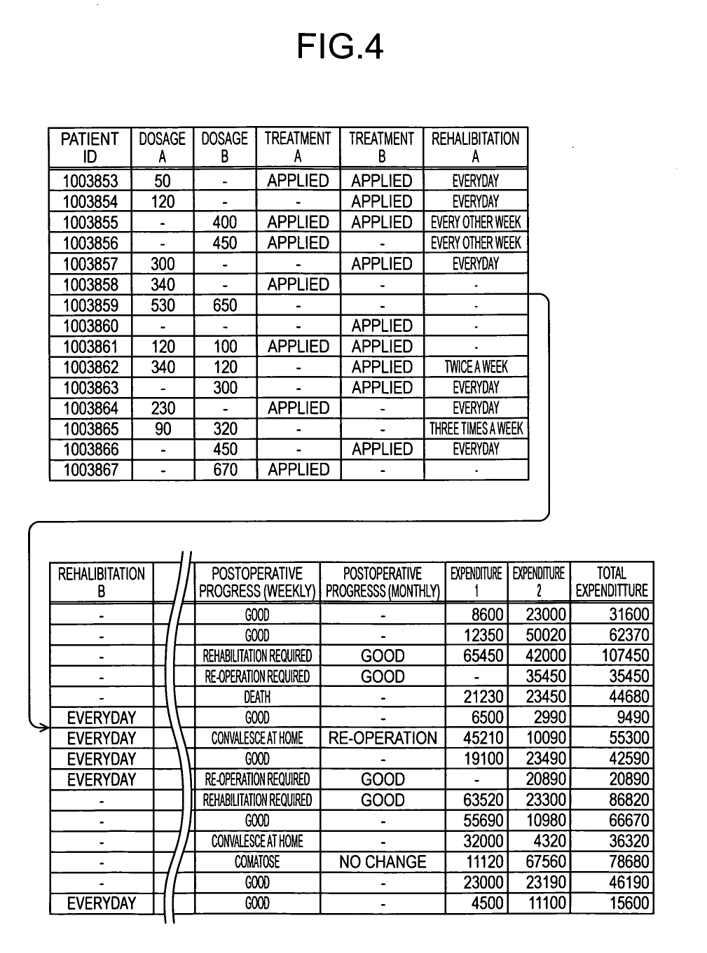

FIG. 4 is a table showing an example of a data structure of the medical treatment data table for recording the history of medical treatment particularly in the case of undergoing the operation among the variety of medical data tables. AS shown in FIG. 4, this medical treatment data table is structured of a plurality of records each created for every patient. Each record contains items (fields) such as a patient ID of the patient, a content of dosage (a dosage A, a dosage B) per medical treatment, a distinction whether each treatment is applied or not (a treatment A, a treatment B), an instruction content of rehabilitative method (a rehabilitation A, a rehabilitation B), a progress after one week since the operation (a postoperative weekly progress), a progress after one month since the operation (a postoperative monthly-progress), an expenditure per medical treatment (an expenditure 1, an expenditure 2), and a total of expenditures.

The hard disk 14 is further stored with a analysis data creation program 31, a case data creation program 32, a medical treatment data creation program 33, an analysis database 42, a case database 43 and a medical treatment database 44 in order to embody the diagnostic support system of the present invention.

The analysis data creation program 31 is executed periodically (at an interval of, e.g., one month, one week, or everyday) or as occasion demands. The analysis data creation program 31 is a program for extracting the records of the respective tables constituting the computerized medical record database 41, generating analysis data by linking the values in necessary fields in each record with the patient ID used as a key, and registering the thus generated analysis data in the analysis database 42. Further, the case data creation program 32 is executed periodically or as occasion demands. The case data creation program 32 is a program for extracting only the values in the fields that are used as the MBR-based case data on the occasion of predicting an actual disease name by executing a cross-search across the respective records in the analysis database 42 in a way that utilizes the aforementioned MBR algorithm, and for registering the extracted case data in the case database 43. Moreover, the medical treatment data creation program 33 is executed periodically or as occasion demands. The medical treatment data creation program 33 is a program for extracting the respective records out of the medical data tables constituting the computerized medical record database 41, and registering the extracted records as medical treatment data in the medical treatment database 44.

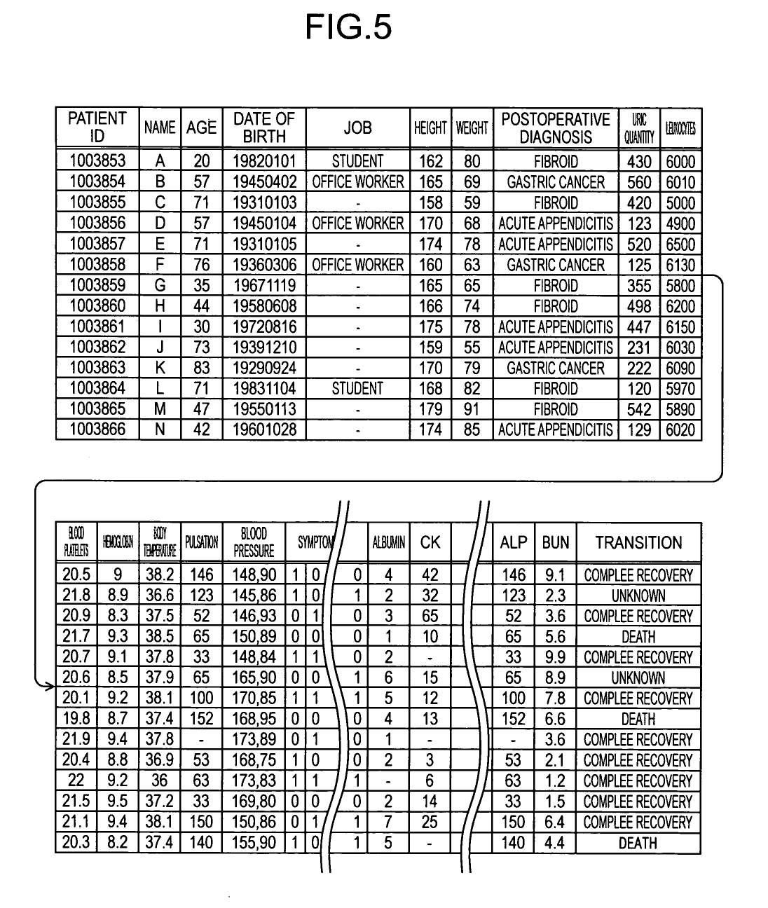

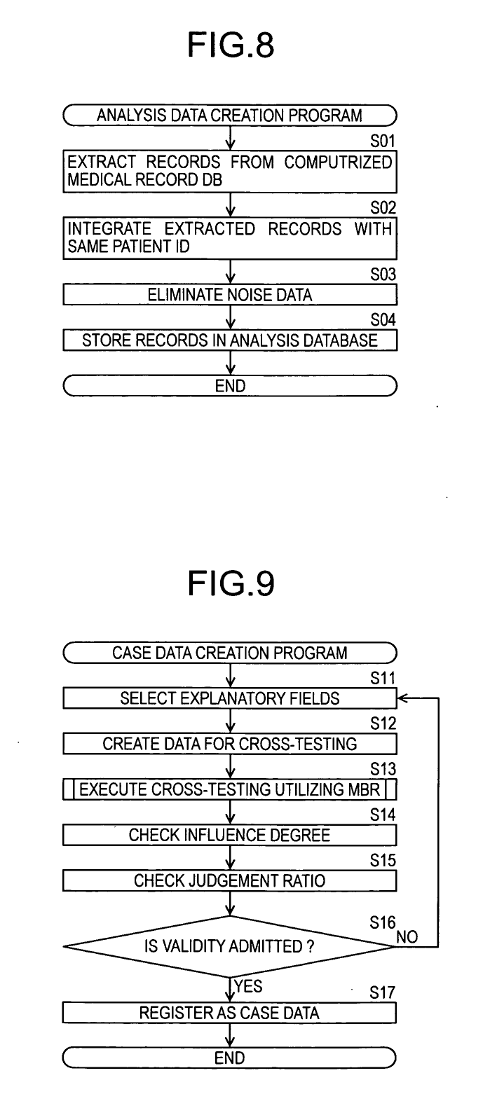

Referring to tables in FIGS. 5 through 7 and flowcharts in FIGS. 8 through 12, contents of specific processes of the respective programs 31 through 33 will hereinafter be explained in detail together with the data structures of the databases 42 through 43 in which the data are to be registered as these programs are executed.

<Analysis Data creation Program and Analysis Database>

To begin with, a content of a process executed by the CPU 10 in accordance with the analysis data creation program 31, will be described referring to FIG. 8.

In first step S01 after starting this process, the CPU 10 extracts all the records recorded in the respective tables of the computerized medical record database 41.

In next step S03, the CPU 10 integrates records of tables extracted in S01 registered with same patient ID. To be specific, the CPU 10 generates, for every patient ID, one aggregated record by joining the patient ID and the values in the respective fields such as the name, the age, the date of birth, the job, the height, the weight and the symptom in the record specified by the patient ID that is extracted from the patient profile data table; the disease name (the postoperative diagnosed disease name) and the variety of examination values in the fields (such as the uric quantity, the leukocytes, the blood platelets, the hemoglobin, the body temperature, the pulsation, the blood pressure, the albumin, the CK, the ALP, the BUN, etc.) in the record specified by the patient ID that is extracted from the examination data table; and a value of a postoperative progress (transition) in the record specified by the patient ID that is extracted from the attending data table.

In next step S03, the CPU 10 eliminates noise data. To be concrete, the CPU 10 compares, for every record generated in S03, the examination values in the respective fields in the concerned record with upper and lower limit values (the values that are too high and too low to be plausible as far as the examinee is alive) prepared beforehand in relation to the respective items. The CPU 10 eliminates, as the noise data, the examination values that do not fall in between the upper and lower limit values corresponding thereto. Further, the CPU 10 compares, for every record, the examination values in the respective fields in that record with the examination values in the same fields in other records, and eliminates as the noise data the examination values that obviously prove to be peculiar as compared with other records.

In next step S04, the CPU 10 stores the processed records in the analysis database 42. FIG. 5 is a table showing a data structure of the analysis database 42 in which the records have thus been registered. upon a completion of the processing in S04, the CPU 10 terminates the processing based on this analysis data creation program 31.

<Case Data creation Program and case Database>

Next, a content of the process executed by the CPU 10 in accordance with the case data creation program 32, will be explained referring to FIG. 9.

In first step S11 after starting this process, the CPU 10 selects explanatory fields (items) from the respective data fields (items) in the analysis database 42. To be specific, the CPU 10 selects, as the explanatory fields, all the fields other than the fields whose values are lacked in many records, the fields insignificant to the MBR (such as the patient ID, the date, etc.)and one of a pair of fields exhibiting a large correlation (e.g., a correlation rate is equal to or larger than 0.9) between their values in the many records. Then, the CPU 10 eliminates all the fields other than the explanatory fields in the analysis database 42.

In next step S12, the CPU 10 extracts all the records in the analysis database 42, and sorts out the records into, e.g., ten groups. Then, the CPU 10 creates ten sets of cross-testing data in each of which records belonging to one group are set as the records for verification, and records belonging to other nine groups are set as case records.

In next step s13, the CPU 10 fetches piece by piece the ten sets of cross-testing data created in S12, and executes the cross-testing for each set of cross-testing data.

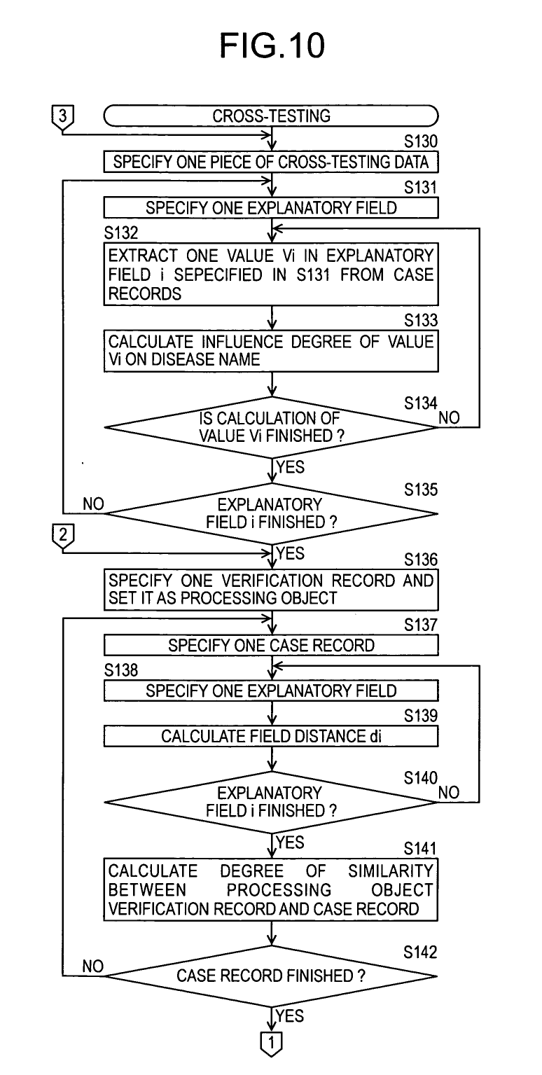

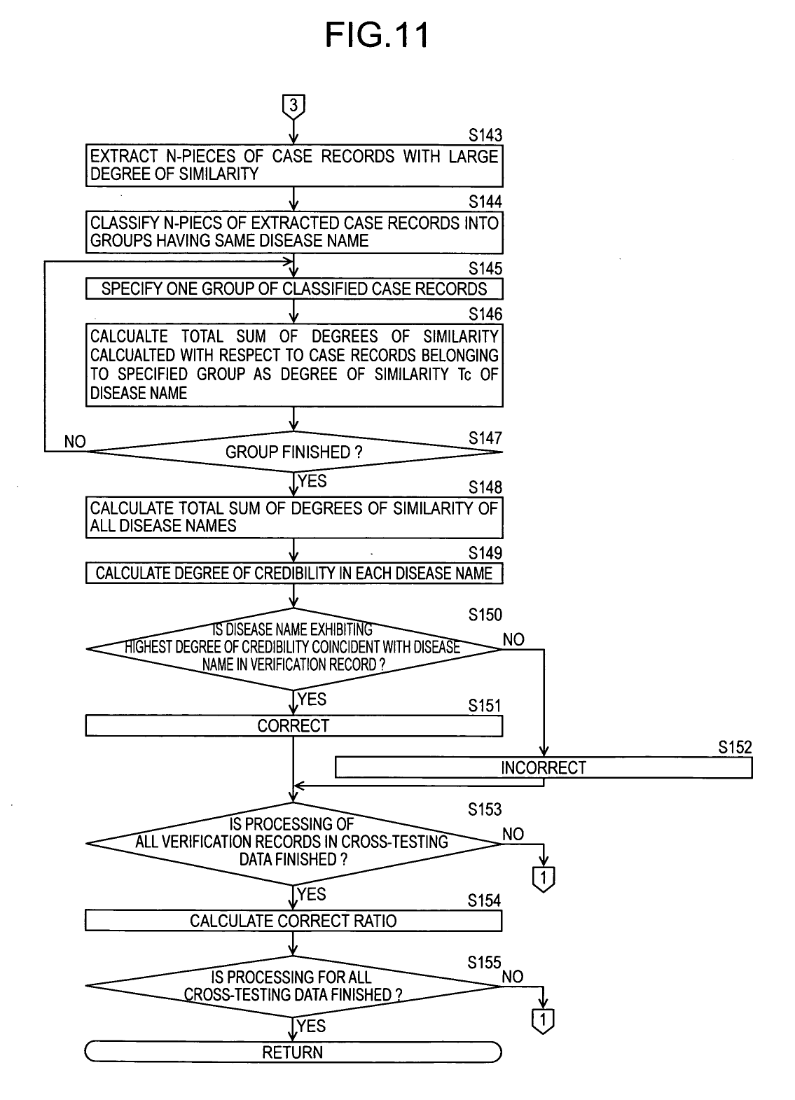

FIGS. 10 and 11 are flowcharts showing a cross-testing process subroutine executed in s13. In first step S130 after entering this subroutine, the CPU 10 specifies one piece of cross-testing data created in S12. upon a completion of the processing in S130, the CPU 10 advances the processing to S131.

In S131, the CPU 10 specifies one of the explanatory fields selected in S11. upon a completion of the processing in S131, the CPU 10 advances the processing to S132.

In S132, the CPU 10 extracts one of values Vi in the, explanatory field i specified in S131 from the case records contained in the cross-testing data specified in S130.

In next step S133, the CPU 10 calculates, as an influence degree Wi of the value Vi, how much the value Vi in the explanatory field i extracted in S132 contributes to determine a disease name (the postoperative diagnosed disease name). More specifically, the CPU 10 calculates a conditioned probability (P(Ck/Vi) showing how much a disease name Ck is biased to the value Vi in the explanatory field i (which is a probability that the disease name is Ck when the value in the field i is Vi) with respect to all of disease names (C1 through Cn). Then, the CPU 10 calculates its square total sum (ΣP(Ck/Vi)²) as the influence degree Wi of the value Vi in the field i. For example, an influence degree of how much a value “20-year old” in the “age” field contributes to determine a disease name, is given by:

Influence degree =P(acute appendicitis/20-year old)²+P(fibroid/20-year old)²+ . . . +(gastric cancer/20-year old)².

In this case, if all the 20-year old persons have the acute appendicitis and have none of other disease names, the influence degree-is given such as:

$$\begin{matrix}\text{Influence degree} = 1.0 + 0.0 + 0.0 + 0.0 + … + 0.0 \\ = 1.0\end{matrix}$$

As can be understood from this example of the calculation, if a disease name Ck is largely biased to the value Vi, the influence degree Wi comes to the maximum.

In next step S134, the CPU 10 checks whether the extraction of all the values Vi in the explanatory field i specified in S131 from the case records contained in the cross-testing data specified in S130, has been completed through the process in S132 or not. Then, if all the values Vi in this explanatory field i are not yet extracted, the CPU 10 has the processing looped. back to S132. whereas if the extraction of all the values Vi in this explanatory field i has been completed, the CPU 10 advances the processing to S135.

In S135, the CPU 10 checks whether or not all the explanatory-fields selected in S11 have been specified through the process in S131. Then, if all the explanatory fields have not yet being specified, the CPU 10 returns the processing to S131. whereas, if all the explanatory fields have been specified, the CPU 10 advances the processing to S136.

The aforementioned processes in S130 through S135 have been done, whereby the CPU 10 has calculated the influence degree (Wi: the influence degree of the value Vi in the field i) at which the value Vi contributes to the disease name, for every value Vi contained in the case records with respect to all the explanatory fields i.

In S136, the CPU 10 specifies one verification record as a processing object from the cross-testing data specified in S130. upon a completion of the processing in S136, the CPU 10 advances the processing to S137.

In S137, the CPU 10 specifies one case record as a comparison object from within the cross-testing data specified in S130. Upon a completion of the processing S137, the CPU 10 advances the processing to S138.

In S138, the CPU 10 specifies one of the explanatory fields specified in S11.

In next step S139, the CPU 10 calculates a degree of similarity between the verification record specified as the processing object in S136 and the case record specified as the comparison object in S137. Namely, the CPU 10 compares a value ui in the explanatory field i specified in S131 within the processing object verification record with the value Vi in the same explanatory field i in the comparison object case record, and calculates a field distance di as follows:

di=|Vi-Ui|/standard deviation of values in explanatory field i (in a case where a numerical value variable is to be set in the explanatory field i)

di=1 (in a case where a category variable is to be set in the explanatory field i and Vi≠Ui)

di=0 (in a case where the category variable is to be set in the explanatory field i and Vi=Ui)

In next step s140, the CPU 10 checks whether or not all the explanatory fields selected in S11 have been specified through the process in S138. Then, if all the explanatory fields have not yet being specified, the CPU 10 returns the processing to S138. whereas if all the explanatory fields have been specified, the CPU 10 advances the processing to S141.

In S141, the CPU 10 calculates a degree of similarity between the processing object verification record and the comparison object case record. To be specific, the CPU 10 weights, for every explanatory field i, a square of the field distance di calculated in S139 by multiplying it by the influence degree Wi of the value Ui in the processing object verification record used for the calculation thereof. Then, the CPU 10 executes the following function for the field distance di with respect to all the explanatory fields i that have been weighted, thereby calculating, as a solution of this function, a degree of similarity (distance) between the verification record and the comparison object case record.

Degree of similarity=1/√[Σ(wi×di²)]

For instance, in a case where in the verification record, a value in the “age” field is “20-year old”, a value in the “leukocytes” field is “6000”, a value in the “blood platelets” field is “20.5”, a value in the field “hemoglobin” is “9”, a value in the “stomachache” is “yes” and a value in the “nausea” field is “no”, the CPU 10 calculates the degree of similarity such as:

Degree of similarity=1/√{((influence degree of age of 20-year old)×(age's field distance²))+((influence degree of leukocytes of 6000)×(leukocyte's field distance²))+((influence degree of blood platelets of 20.5)×(blood platelet's field distance²))+((influence degree of hemoglobin of 9)×(hemoglobin's field distance²))+((influence degree of there being stomachache)×(stomachache's field distance²))+((influence degree of there being no nausea)×(nausea field distance²))+ . . . }

Upon a completion of the processing in S141, the CPU 10 advances the processing to S142.

In S142, the CPU 10 checks whether or not all the case records in the cross-testing data specified in S130 have been specified through the process in S137. Then, if all the case records have not yet being specified, the CPU 10 returns the processing to S137. whereas if all the case records have being specified, the CPU 10 advances the processing to S143.

The aforementioned processes in S136 through S142 enable the CPU 10 to calculate the degree of similarity respectively between each of the case records contained in the cross-testing data specified in S130 and the processing object record.

In S143, the CPU 10 extracts N-pieces (N: a preset integer) of case records in sequence from the record having the largest similarity to the processing object record down to the smallest.

In next step S144, the CPU 10 classifies the N-pieces of case records extracted in S143 into groups of records having same value (the same disease name) in the “disease name (postoperative diagnosed disease name)” field. upon a completion of the processing in S144, the CPU 10 makes the processing proceed to S145.

In S145, the CPU 10 specifies one group out of the groups of the case records classified in S144.

In next step S146, the CPU 10 calculates a total sum of the degrees of similarity of the processing object verification record calculated with respect to all the case records belonging to the group specified in S145, as a degree of similarity Tc of the disease name common throughout this group.

In next step S147, the CPU 10 checks whether or not all the groups classified in S144 have being specified through the process in S145. Then, if all the groups have not yet being specified, the CPU 10 returns the processing to S145. whereas if all the groups have being specified, the CPU 10 advances the processing to S148.

Through the processes in S144 to S147, the CPU can calculate the degree of similarity Tc with respect to all the disease names.

In S148, the CPU 10 calculates a total sum of the degrees of similarity Tc which have been calculated with respect to all the disease names.

In next step S149, the CPU 10 calculates, as a degree of credibility in each of the disease names, a ratio of the degree of similarity Tc of each disease name to the total sum of the degrees of similarity Tc that have been calculated in S148.

In next step S150, the CPU 10 checks whether the disease name exhibiting the highest degree of credibility calculated in S149 is coincident with a value (disease name) in the “disease name (postoperative diagnosed disease name)” field in the processing object verification record. Then, if they are coincident with each other, the CPU 10 records, on the RAM 12, a check result of “correct” concerning the processing object verification record in S151, and advances the processing to S153. In contrast, if they are not coincident with each other, the CPU 10 records on the RAM 12 a check result of “incorrect” with respect to the processing object verification record in S152, and advances the processing to S153.

In S153, the CPU 10 checks whether or not-all the verification records in the cross-testing data specified in S130 have being specified through the process in S136. Then, if all the verification records have not yet being specified, the CPU 10 returns the processing to S136. whereas if all the verification records have being specified, the CPU 10 advances the processing to S154.

The above processes in S136 through S153 enable the CPU 10 to acquire the check result of “correct” or “incorrect” in regard to all the verification records contained in the cross-testing data specified in S130.

In S154, the CPU 10 calculates, as a correct ratio, a ratio of number of verification records. recorded with “correct” to total number of verification records contained in the cross-testing data specified in S130.

In next step S155, the CPU 10 checks whether or not all the cross-testing data created in S12 have being specified through the process in S130. Then, if all the cross-testing data have not yet being specified, the CPU 10 returns the processing to S130. whereas if all the cross-testing data have being specified, the CPU 10 terminates this cross-testing process.

As discussed above, the CPU 10 acquires ten pieces of correct ratio on the basis of all of ten sets of cross-testing data by completing the cross-testing process in s13.

In next step S14, the CPU 10 checks the influence degree. Namely, in the cross-testing process in s13, the influence value Wi for every value Vi in each examination field i is calculated each time the processes in S133 through S135 are executed based on each cross-testing data (S133). Accordingly, ten pieces of influence values Wi at the maximum are calculated with respect to each value Vi. The CPU 10 calculates, for every value Vi, an average of all the influence degrees Wi calculated in connection with the value Vi. Then, the CPU 10 checks whether or not there is a value Vi for which an extremely large average value of the influence values Wi is calculated, whether or not there is a value Vi for which an extremely small average value of the influence values Wi is calculated, and whether or not the number of the influence degrees Wi is well balanced with the calculated average value (whether or not there is a field value Vi for which a low average value of the influence degrees Wi was calculated although the number of cases is large, and whether or not there is a field value Vi for which a high average value of the influence degrees Wi was calculated although the number of cases is small).

In next step S15, the CPU 10 calculates an average value of ten pieces of correct ratios calculated for every piece of cross-testing data in s13, and checks this average value. Namely, the CPU 10 checks whether the average value of the correct ratios is extremely low or not.

In next step S16, the CPU 10 judges based on the check results in S14 and S15 whether or not a validity is admitted in the records in the analysis database 42 at a point of the present time. Then, the CPU 10 deems that the validity is not admitted if there is the value Vi for which the extremely large average value of the influence degrees Wi is calculated, if there is the value Vi for which the extremely small average value of the influence degrees Wi is calculated, if there is the value Vi for which the low average value of the influence degrees Wi is calculated although the number of cases is large, if there is the value Vi for which the high average value of the influence degrees Wi is calculated although the number of cases is small, or if the average value of the correct ratio is extremely low. Then, the CPU 10 returns the processing to S11, and deletes the values exerting a peculiar influence on the average value of the correct ratio (that is, the value Vi for which the extremely large average value of the influence degrees Wi is calculated, the value Vi for which the extremely small average value of the influence degrees Wi is calculated, the value Vi for which the low average value of the influence degrees Wi is calculated although the number of cases is large, and the value Vi for which the high average value of the influence degrees Wi is calculated although the number of cases is small). The CPU 10 also deletes the unnecessary explanatory fields i themselves that have less of the residual values Vi as a result of that deletion, and thereby modifies the analysis database 42. Then, the CPU 10 re-executes the processes from S12 onwards. By contrast, if there is neither the value Vi for which the extremely large average value of the influence degrees Wi is calculated, nor the value Vi for which the extremely small average value of the influence values Wi is calculated, nor the field value Vi for which the low average value of the influence degrees Wi is calculated although the number of cases is large, nor the field value Vi for which the high average value of the influence degrees Wi is calculated although the number of cases is small and the average value of the correct ratio is equal to or larger than a predetermined value, the CPU 10 deems that the validity is admitted, and hence advances the processing to S17.

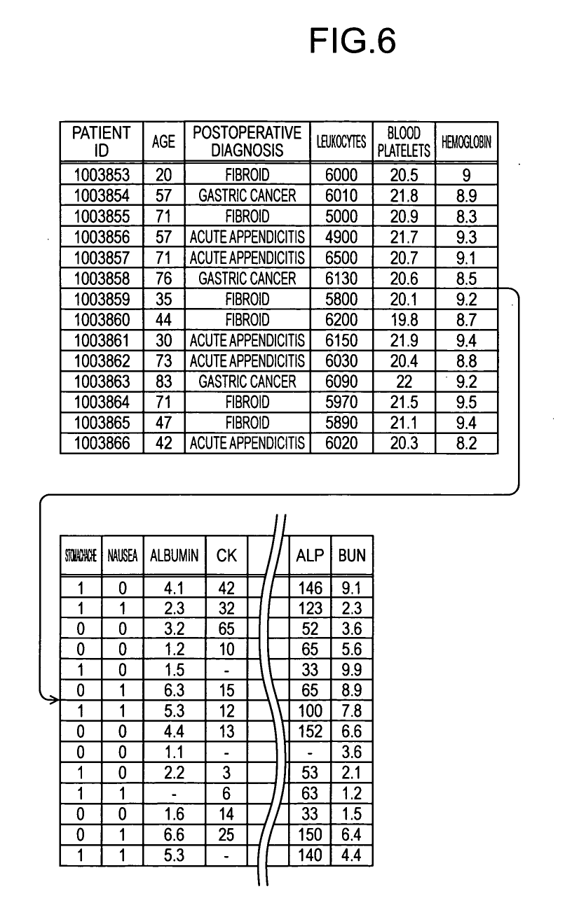

In S17, the CPU 10 stores the case database 43 with the respective records left on the analysis database 42, as the case data. FIG. 6 is a table showing a data structure of the case database 43 on which the records are thus registered. Namely, the case database 43 comes to becomes equivalent to a case database accumulated with multiple pieces of case data constructed of the values in the predetermined plural number of fields with respect to symptoms of past patients. upon a completion of the processing in S17, the CPU 10 terminates the processes based on this case data creation program 32.

<Medical Treatment Data creation Program and Medical Treatment Database>

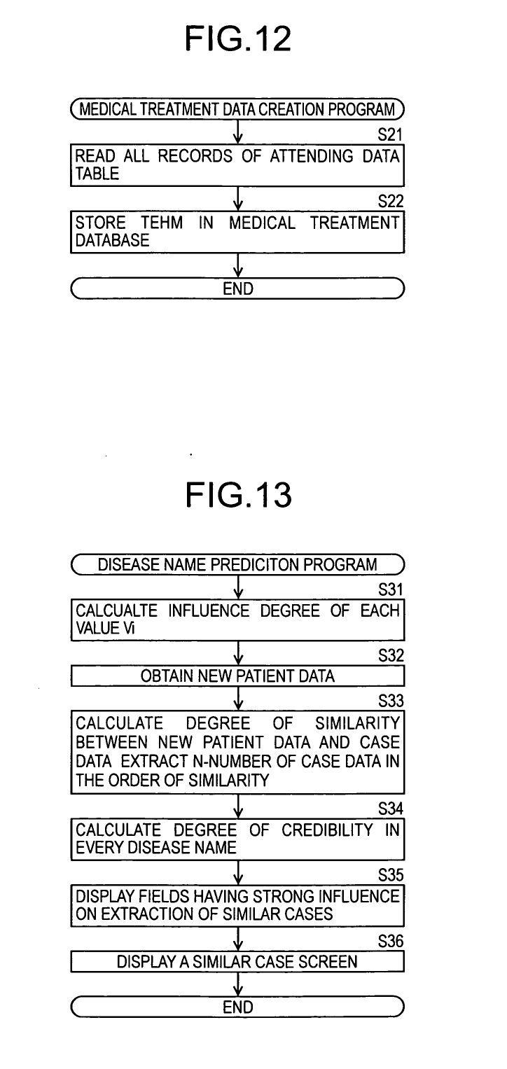

Next, a content of the process executed by the CPU 10 in accordance with the medical treatment data creation program 33, will be explained with reference to FIG. 12.

In first step S21 after starting this process, the CPU 10 reads all the records of the attending data table out of the computerized medical record database 41.

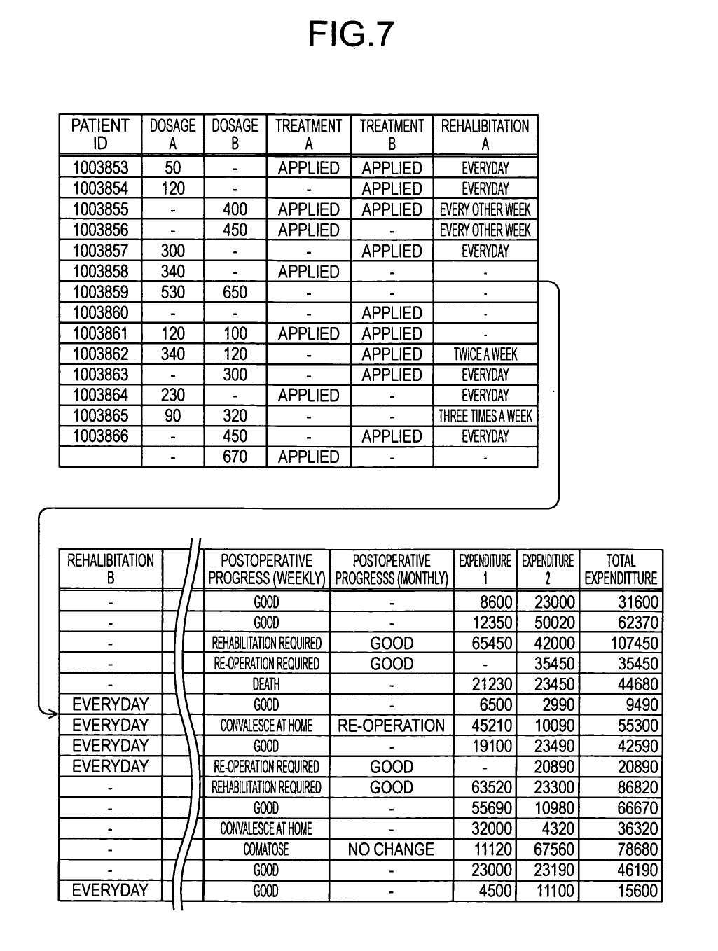

In next step S22, the CPU 10 stores the medical treatment database 44 with all the record read out in S21. FIG. 7 is a table showing a data structure of the medical treatment database 44 on which the records are thus registered. upon a completion of the processing in S22, the CPU 10 terminates the process based on this medical treatment data creation program 33.

(Terminal)

Each terminal 2 is constructed of a CPU (central Processing unit) 20 as a processing device for controlling the whole device, and an input device 21, a RAM (Random Access memory) 22, a display 23, a hard disk 24 and an interface adapter 25 which are connected via a bus B to this CPU 20. Among these components, the input device 21 is constructed of a keyboard and a mouse that are operated by an operator to input commands and data to the CPU 20. Further, the RAM 22 is a main memory device on which an operation area utilized by the CPU 20 is developed, and the display 23 is a display device for displaying screens generated by the CPU 20. Moreover, the interface adapter 25 is defined as a LAN card serving as an interface with the computer network N and is thereby utilized for accessing the hard disk 14 of the host computer 1 via a signal line.,

The hard disk 24 is a computer readable storage medium such as a hard disk, etc. stored with the variety of programs and the various categories of data. As explained above, this terminal 2 is the developed version of the terminal in the conventional computerized medical record system, and hence the hard disk 24 is preinstalled with an unillustrated computerized medical record client program in addition to an unillustrated os (operating system) that supports a communication function via the computer network N. The unillustrated computerized medical record client program instructs the CPU 20 to send a retrieval request containing a retrieval condition inputted via the input device 21 to the host computer 1, to display a content of the data responded from the host computer in response to this retrieval request on the display 22 in a way that embeds the same data content into a computerized medical record display screen in a predetermined format, and to transmit data created afresh and data updated on this computerized medical record display screen to the host computer 1.

The hard disk 24 is further preinstalled with a disease name prediction program 51 and an information display program 52 in order to embody the diagnostic support system of the present invention.

The disease name prediction program 51 is a program for reading, from the case database, records exhibiting a high degree of similarity to data (examination values, a complaint of the patient, an observation of the examination) about symptoms of a new patient that are inputted through the input device 21. The disease name prediction program 51 is the program for calculating a degree of credibility in every disease name in these records, displaying these disease names together with the degrees of credibility thereof on the display 23, and also displaying an influence degree, as a ground of presuming the disease name, for every value in each field concerning the symptom of the new patient which has been used for calculating the degree of similarity. The information display program 52 is a program for displaying a history of medical treatment and an expenditure corresponding to each record exhibiting the high degree of similarity in accordance with an instruction inputted via the input device 21 by the operator who has seen the data displayed on the display 23 by the disease name prediction program 51.

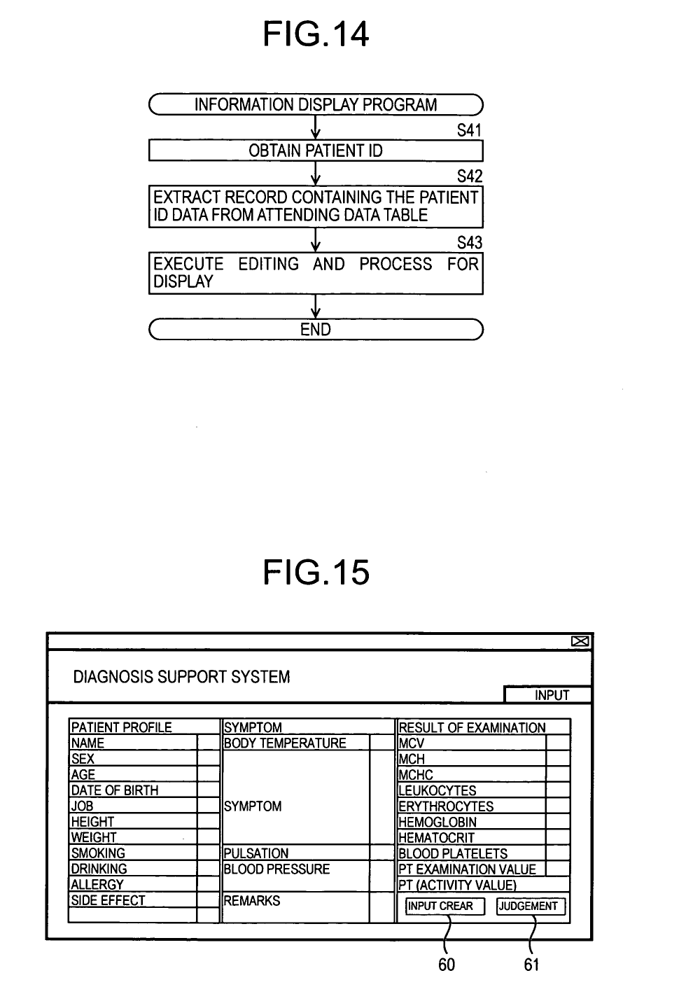

Specific contents of the processes of the programs 51 and 52 will be discussed in detail with reference to flowcharts in FIGS. 13 and 14 and examples of screens shown in FIGS. 15 through 18.

<Disease Name Prediction Program>

To begin with, a content of the process executed by the CPU 20 in accordance with the disease name prediction program 51 will be explained referring to FIG. 13.

In first step S31 after starting this process, the CPU 20 accesses the host computer 1 and reads contents of the case database 43. Then, the CPU 20 calculates, as an influence degree Wi of a value Vi, how much the value Vi contributes to determine a disease name (a postoperative diagnosed disease name) with respect to every value Vi with respect to all the explanatory fields (which are data fields other than the patient ID) i in the case database.43. Namely, the CPU 20 calculates, per value Vi, a conditioned probability (P(Ck/Vi) showing how much a disease name Ck is biased to the value Vi with respect to all of disease names (C1 through Cn). Then, the CPU 10 calculates its square total sum (ΣP(Ck/Vi)²) as the influence degree Wi of the value Vi.

In next step S32, the CPU 10 displays an input screen shown in FIG. 15 on the display 23, and executes a process for acquiring data of the new patient for prediction of disease name. This input screen is linked to the unillustrated computerized medical record client program and thus serves as an input screen for the computerized medical record data. This screen therefore contains input boxes for all the data fields in the patient profile data table and the examination data table in the computerized medical record database 41. Further, this input screen is provided with an “input clear” button 60 and a “decision” button 61. when the operator, presses the “decision” button 61 in a state where the various pieces of data of new patient are inputted to the respective input boxes corresponding thereto, all the values inputted to the input boxes are requested by the unillustrated computerized medical record client program to be registered in the computerized medical record database 41. At the same time, all the values are captured as new patient data by the disease name prediction program 51, and the processing proceeds to S33.

In S33, the CPU 10 calculates a degree of similarity between the new patient data and case data of each record (which will hereinafter be called [case data]) stored in the case database 43. Namely, the CPU 10 compares, for every explanatory field i composing the case data, the value Vi in the explanatory field i with the value Ui in the same field in the new patient data, and calculates a field distance di as follows:

di=|Vi-Ui|/standard deviation of values in explanatory field i (in a case where a numerical value variable is to be set in the explanatory field i)

di=1 (in a case where a category variable is to be set in the explanatory field i and Vi≠Ui)

di=0 (in a case where the category variable is to be set in the explanatory field i and Vi=Ui)

Moreover, the CPU 10 multiplies a square of the field distance di calculated for each explanatory field i by the influence degree Wi of the value Ui in the new patient data used for the calculation thereof, and thereby effects weighting. Then, the CPU 10 executes the following function for the field distances di with respect to all the explanatory fields i that have undergone weighting, thereby calculating, as a solution of this function, a degree of similarity (distance) between the new patient data and the comparison object case data.

Degree of similarity=1/√[Σ(wi×di²)]

Thus, the CPU 10 calculates the degree of similarity of each piece of case data to the new patient data on the basis of a value aggregation obtained by weighting a difference between a value of the case data and a value of the new patient data for every field according to an influence degree of the same value of the new patient data. then, the CPU 10 extracts, as examples of similar case, N-pieces (N: a preset integer) of case data in sequence from the data having the largest similarity to the thus-calculated new patient data down to the smallest.

In next step S34, the CPU 10 calculates a degree of credibility of every disease name on the basis of the value (disease name) in the “disease name (postoperative diagnosed disease name)” field in the N-pieces of similar cases extracted in S33. Namely, the CPU 10 classifies the N-pieces of extracted similar cases into groups having the same disease name. Then, the CPU 10 calculates a total sum of the degrees of similarity calculated with respect to all the similar cases in each group, as a degree of similarity Tc of the disease name. The CPU 10 calculates a ratio of the degree of similarity Tc of each disease name with respect to the total sum of the thus calculated degrees of similarity Tc of all the disease names, as a degree of credibility Ac in the disease name.

In next step S35, the CPU 10 specifies the fields strongly influencing on extraction of the similar cases in sequence from the highest influence degree down to the lowest as a ground for judging a disease name of the new patient. Namely, the CPU 10 specifies the influence degrees Wi (the influence degrees Wi of values Ui in the new patient data with respect to the same explanatory fields i as those composing the case data) of the values Ui in the respective fields i in the new patient data used for calculating the degree of similarity in S33, in sequence from the largest influence degree down to the smallest.

Then, the CPU 10 displays, on the display 23, a disease confirmation screen for presenting the influence degree Wi of the value Ui with respect to each explanatory field i specified and the degree of credibility Ac in each disease name that is calculated in S34.

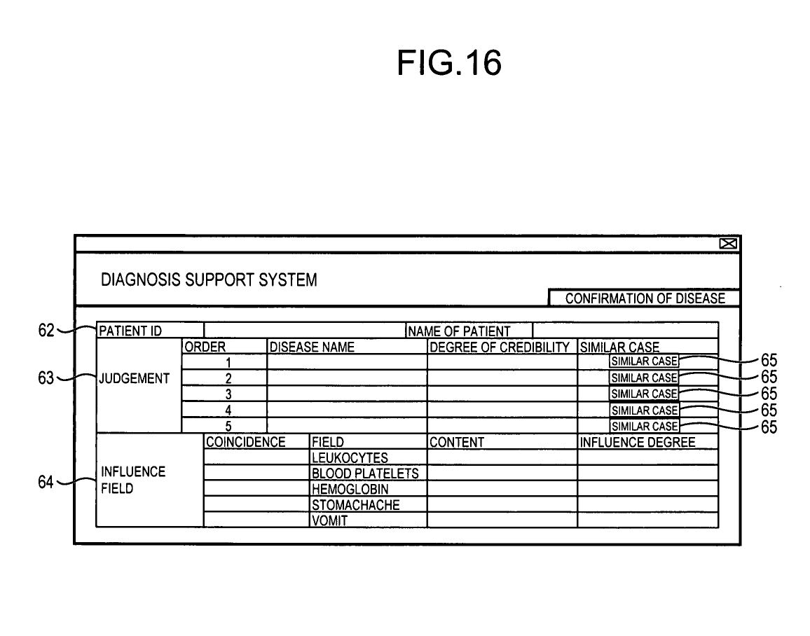

As shown in FIG. 16, this disease confirmation screen consists of a patient identification box 62, a judgement box 63 and an influence field box 64 laid out vertically. The patient identification box 62 is structured of one line, wherein a patient ID and a patient name of the new patient are so displayed as to be arranged side by side.

The judgement box 63 is structured of a plurality of lines (five lines in an example in FIG. 16) corresponding to the respective disease names and vertically arranged in sequence from the disease name exhibiting the highest degree of credibility Ac, wherein, each line contains sequentially from the left an “order” field indicating a degree of credibility Ac of the disease name, a “disease name” field indicating the disease name, a “degree of credibility” field indicating a degree of credibility Ac in the disease name and a “similar case” field indicating a similar case button 65.

The influence field box 64 is structured of N-lines (five lines in the example in FIG. 16) corresponding to the influence degrees Wi of the values Ui with respect to the individual explanatory fields i specified as described above and arranged vertically in sequence from the highest influence degree wi, wherein each line contains sequentially from the left a “coincidence” field indicating coincidence that will be explained later on, an “explanatory field” field indicating the explanatory field i, a “value” field indicating a value Ui thereof, and an “influence degree” field indicating the influence degree Wi thereof. The “coincidence” field in each line is a field for showing, with a mark (which is a form corresponding to a fact) (a black circle represents “coincidence” in the example in FIG. 16), whether or not there is any similar case having the disease name displayed at first time in the judgement box 63 and containing a value Vi substantially coincident with the value Ui displayed in the “value” field in same line.

When the operator inputs an instruction of displaying the similar case data by pressing any one of the “similar case” buttons 65 in the judgement box 63 by use of the input device 21, the CPU 20 advances the processing to S36.

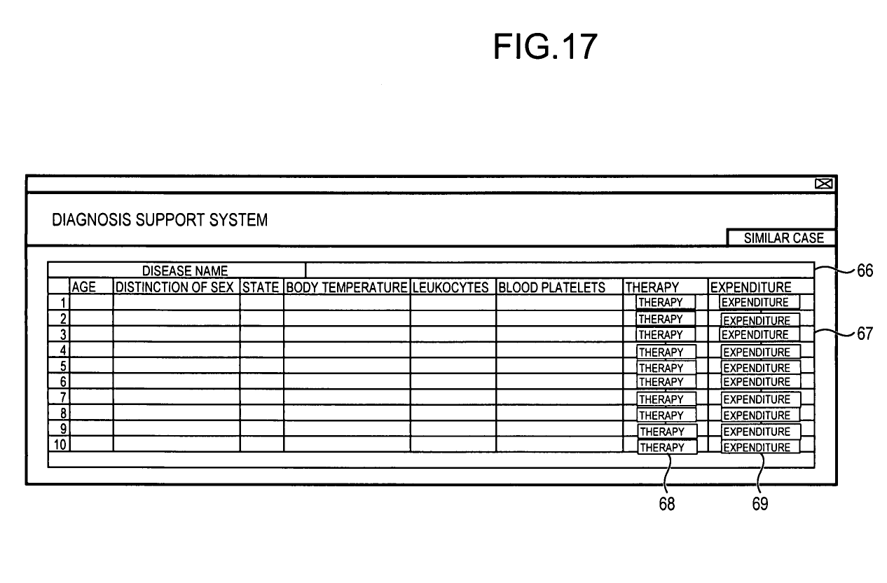

In S36, the CPU 20 displays, on the display 23, a similar case screen for showing contents of the similar cases as a ground-for presuming the relevant disease name-on the basis of the similar cases containing the disease name displayed in the same line as the pressed “similar case” button 64 is displayed in the judgement box 63. As illustrated in FIG. 17, this similar case screen consists of a disease name display box 66 for displaying the relevant disease name and a similar case box 67 laid out vertically. The similar case box 67 is structured of a plurality of lines (ten lines in an example in FIG. 17) corresponding to respective similar cases and vertically arranged in sequence from the case exhibiting the highest degree of similarity down to the lowest, wherein each line contains an “age” field, a “sex” field, a “state” field (corresponding to the “transition” field in FIG. 5), a “body temperature” field, some fields (a “leukocytes” field and a “blood platelets” field in the example in FIG. 17) corresponding to the fields attached with the mark in the “coincidence” field in the influence field box 64 on the disease confirmation screen, a “treatment” field indicating a treatment button 68 and an “expenditure” field indicating an expenditure button 69. when the operator presses any one of the “treatment” buttons 68 or the “expenditure” buttons 69 in the similar case box 67 by use of the input device 21, the CPU 20 boots the information display program 52.

<Information Display Program>

Next, a content of the process executed by the CPU 20 in accordance with the information display program 52 booted as described above, will be explained referring to FIG. 14.

In first step S41 after starting this process, the CPU 20 acquires, from the disease name prediction program 51, types of the buttons 68 or 69 pressed on the similar case screen and a patient ID in the similar case corresponding to the buttons 68 or 69.

In next step S42, the CPU 20 accesses the host computer 1, and extracts a record containing the patient ID transferred from the disease name prediction program 51, out of the attending data table in the computerized medical record database 41.

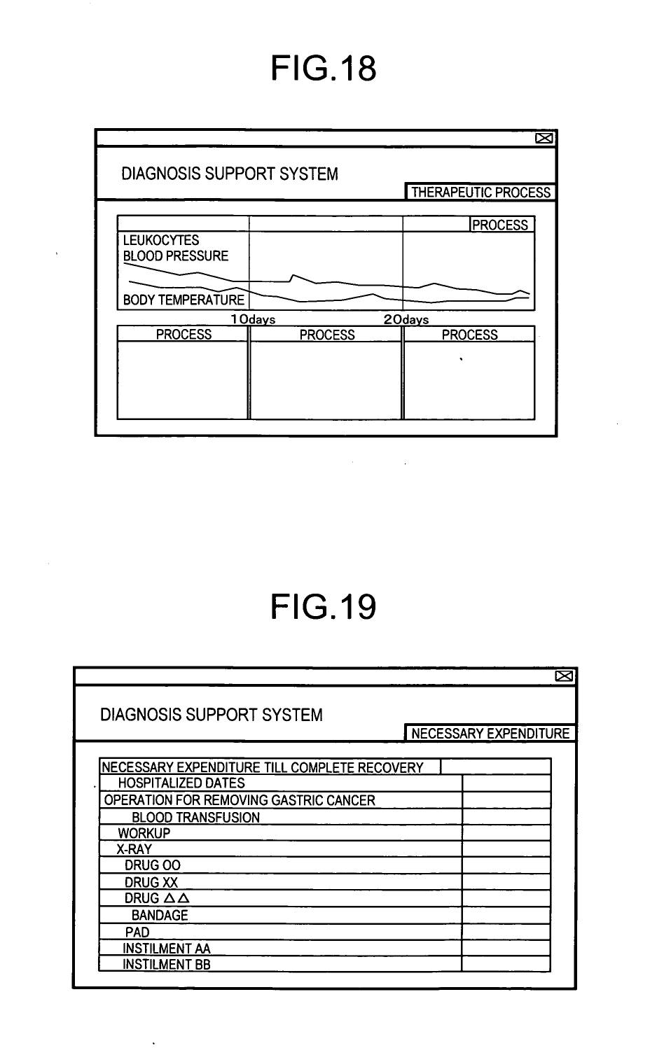

In next S43, the CPU 20 edits and processes, for displaying the screen, contents of the record containing the relevant patient ID extracted from the attending data table in S42. Namely, the CPU 20, when the “treatment” button 68 is pressed, edits and processes the data in the respective fields other than the “expenditure” field in the relevant record into a format for a medical treatment process screen shown in FIG. 18. Further, when the “expenditure” button 69 is pressed, the CPU 20 edits and processes the data in the .“expenditure” field in the relevant record into a format for a necessary expenditure screen illustrated in FIG. 19. Then, each of the screens generated by the editing and processing is displayed on the display 23.

(Operation of the Embodiment)

According to the diagnosis support system in the embodiment having the architecture described above, the values in the respective explanatory fields about the past patient's symptoms used for judging the disease name are processed in a state where the validity is previously assured (a state where the field values not serving for any reference because of being peculiar to the individual patient are eliminated, the fields considered unnecessary for the reason such as being vacant in many case data, etc. are eliminated, and it is therefor actually proved that-the correct disease name can be drawn out at a predetermined or higher probability), and are stored as the case data in the case database 43 (FIG. 9).

Then, when the operator starts up the disease name prediction program 51, the influence degree Wi of the value Vi, how much the value Vi contributes to determine the disease name, is calculated for every value Vi in each of the explanatory fields i stored in the case database 43 (S31). Then, when the operator inputs pieces of new patient data (a patient profile, examination values, a complaint of the patient, an observation of the examination) pertaining to the new patient to the terminal 2 (S32), degrees of similarity of respective pieces of case data stored on the case database 43 to the new patient data are calculated as a total sum of products of the influence degrees Wi of the values Ui of the new patient data with respect to a square of the distance (the field distance di) between the value Vi in the relevant case data for every explanatory field i composing the case data and the value Ui of the new patient data. Then, N-pieces of case data are extracted as similar cases in sequence from the data exhibiting the largest degree of similarity down to the smallest (S33). Then, the similar cases are sorted out into some groups by disease name; the total sum of the degrees of the similarity of case data in each group is calculated as a degree of similarity Tc of the disease name; and a degree of credibility Ac in each disease name is calculated as a ratio of the degree of similarity Tc of each disease name to the total sum of the degrees of similarity Tc in all the disease names (S34). Displayed on the disease confirmation screen (FIG. 16) are the disease names together with their degrees of credibility in sequence from the disease name exhibiting the thus-calculated highest degree of credibility Ac to the smallest. Displayed also on this screen are the values Ui in the respective explanatory fields i in the new patient data that are used for calculating the degree of similarity together with their influence degrees-in sequence from the value exhibiting the highest influence degree Wi down to the lowest. Further, if there exist any similar case having the disease name exhibiting the highest degree of credibility Ac and containing the value Vi in the explanatory field i that is coincident with the value Ui in the new patient data, a mark is given to this value Ui in the explanatory field i (S35).

It is therefore feasible from the operator to know the disease name having a multiplicity of similar cases exhibiting a comparatively large degree of similarity to the new patient data as what could be presumed to be a name of the disease which the new patient suffers from. Moreover, the operator is able to know the value Ui (especially the value Ui in the explanatory field i that is given the mark) in each explanatory field i in the new patient data displayed simultaneously as a ground on which that disease name was presumed. Accordingly, the operator can have a confidence in this presumption by judging himself or herself whether the presumption of the disease name is valid or not.

Further, when the operator presses the “similar case” button 65 corresponding to any one of the disease names displayed on the disease confirmation screen, contents of the similar cases having this disease name are displayed as a list on the similar case screen (FIG. 17) (S36). Accordingly, the operator can verify-by himself or herself the ground of presuming the disease name.

Still further, when the operator presses the “medical treatment” button 68 corresponding to any one of the similar cases displayed on the similar case screen, a history of medical treatment in this similar case is read out of the medical treatment database 44 and displayed (FIG. 14) on a medical treatment process screen (FIG. 18). Therefore, the operator is able to make a treatment plan with reference to the history of medical treatment in the similar case.

According to the present invention having the architecture described above, the name of the disease from which the patient concerned suffers is presumed by collating with the case data accumulated in the past with respect to the symptom of the patient, and the presumed disease name can be displayed together with the ground of this presumption.

We claim:

1. A diagnostic support system, comprising:
a storage device stored with a case database accumulated with multiple pieces of case data including values in a predetermined plural number of fields with respect to symptoms of past patients; and
a computer including an interface, connected to said storage device via a signal line, for accessing said storage device, a processing device, an input device and a display device,
wherein said processing device of said computer: calculates, for every concrete values in respective fields in the case data accumulated in the case database, an influence degree of the value contributing to determine a disease name;
when new patient data including values in some or all of the predetermined fields with respect to a symptom of a new patient are inputted via said input device, calculates a degree of similarity of each piece of case data to the new patient data on the basis of values obtained for respective fields by weighting a difference between a value in each field of the case data and a value in its corresponding field of the new patient data with influence degree of that value in the new patient data;
calculates, for every disease name, a degree of similarity of the disease name on the basis of degrees of similarity in all the case data having this disease name; and
displays on said display device a disease name exhibiting a maximum degree of similarity together with the value in the field in the new patient data of which influence degree is maximum among those used for calculating the degree of similarity.

2. A diagnostic support system according to claim 1, wherein said processing device of said computer displays on said display device a predetermined number of disease names in sequence from the disease name exhibiting the maximum degree of similarity together with their degrees of credibility calculated based on their degrees of similarity.

3. A diagnostic support system according to claim 1, wherein said processing device of said computer displays on said display device values in a predetermined number of fields in the new patient data in sequence from the value exhibiting the maximum influence degree together with their influence degrees.

4. A diagnostic support system according to claim 1, wherein said processing device of said computer, if a value substantially coincident with the value in the new patient data is contained in the case data containing the disease name to be displayed on said display device, displays the value in the field in the new patient data in a special form.

5. A diagnostic support system according to claim 1, wherein said processing device of said computer displays, when an instruction of displaying similar case data with respect to the disease name displayed on said display device is inputted via said input device, contents of predetermined number of pieces of case data in sequence from the content exhibiting the maximum degree of similarity calculated on said display device.

6. A diagnostic support system according to claim 1, wherein said storage device is connected via a computer network linked to an interface of said computer to other computer.

7. A diagnostic support program for a computer comprising an interface for accessing a storage device stored with a case database accumulated with multiple pieces of case data including values in a predetermined plural number of fields with respect to symptoms of past patients, an input device and a display device, said program making said computer execute:
calculate, for every concrete value in respective fields in the case data accumulated in the case database, an influence degree of the value contributing to determine a disease name;
when new patient data including values in some or all of the predetermined fields with respect to a symptom of a new patient are inputted via said input device, calculate a degree of similarity of each piece of case data to the new patient data on the basis of values obtained for respective fields by weighting a difference between a value in each field of the case data and a value in its corresponding field of new patient data with influence degree of that value in the new patient data;
calculate, for every disease name, a degree of similarity of the disease name on the basis of degrees of similarity in all the case data having this disease name; and
display on said display device a disease name exhibiting a maximum degree of similarity together with the value in the field in the new patient data of which influence degree is maximum among those used for calculating the degree of similarity.
